(19) United States
(12) Patent Application Publication
(10) Pub. No.: US 2010/0273240 A1
(43) Pub. Date: Oct. 28, 2010

(54) METHOD FOR PRODUCTION AND PURIFICATION OF MACROMOLECULAR COMPLEXES

(75) Inventor: Suparna Sanyal, Uppsala (SE)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIO-SCIENCES AB, UPPSALA (SE)

(21) Appl. No.: 12/743,573

(22) PCT Filed: Nov. 18, 2008
(86) PCT No.: PCT/SE2008/000645

§ 371 Date: Jun. 15, 2010

(30) Foreign Application Priority Data
Nov. 20, 2007 (SE) 0702575-2

(51) Int. Cl. C07K 14/00; C12N 1/21

(52) U.S. Cl. 435/252.33; 530/395

(57) ABSTRACT

The present invention relates to a method for production and purification of affinity tagged macromolecular complexes, such as ribosomes. More closely, the method comprises in-frame fusion of a nucleotide sequence specific for an affinity tag and a selection marker, wherein the fusion is at the chromosomal site of a gene encoding a multicopy protein, and wherein the macromolecular complex is expressed with multiple copies of said affinity tag. The invention also relates to affinity tagged ribosomes, to cells comprising such affinity tagged ribosomes, and to various uses thereof.

### CROSS-REFERENCE TO RELATED APPLICATIONS

This application is a filing under 35 U.S.C. §371 and claims priority to international patent application number PCT/SE2008/000645 filed Nov. 18, 2008, published on May 28, 2009, as WO 2009/067068, which claims priority to patent application number 0702575-2 filed in Sweden on Nov. 20, 2007.

### FIELD OF THE INVENTION

The present invention relates to a method for production and purification of affinity tagged macromolecular complexes, such as ribosomes. In a preferred embodiment the invention relates to a method to produce affinity tagged ribosomes by inserting the tag at the chromosomal level.

### BACKGROUND OF THE INVENTION

The bacterial ribosome, usually called the 70S ribosome, consists of a large subunit called 50S and a small subunit called 30S, wherein the S stands for the Svedberg, a measure of sedimentation rate. The ribosome comprises at least 50 proteins and three RNAs (5S, 16S and 23S) and is the largest macromolecular assembly of the bacterial cell. There is a growing interest in the optimized system for in vitro synthesis of custom proteins and peptides, which has ribosome as the major component. Most of these methods rely on purification of active ribosome and ribosomal subunit from the bacterial cells, more specifically from Escherichia coli, which is most widely used for the basic research on bacterial protein synthesis. Conventional method of E. coli ribosome purification demands special instrumentation and involves several steps of ultracentrifugation and/or column chromatography, and is therefore quite expensive in terms of time, effort, equipment and reagents. Several attempts have been made to develop a simpler protocol for purification of active ribosomes by different groups without much success.

Affinity tag based purification method revolutionized the protein purification field. Attempts to purify bacterial, plant and yeast ribosomes using affinity tags have been published recently (Gan et al., 2002; Inada et al., 2002; Leonov et al., 2003; Youngman and Green, 2005; Zanetti et al., 2005). Two of these methods employed streptavidin binding aptamer tag (Leonov et al., 2003) and MS2 coat protein binding tag (Youngman and Green, 2005) respectively, fused with the rRNA operon on a plasmid. These two methods were aimed mainly for the purification of E. coli ribosomes bearing mutation in the rRNAs. The other methods involved fusion of either Flag-His₆ tag (Inada et al., 2002; Zanetti et al., 2005) or S-peptide tag (Gan et al., 2002) to some ribosomal protein from Saccharomyces cerevisiae and Arabidopsis thaliana respectively, over-expressed from a plasmid.

JP 2005-261313 describes affinity tagged ribosomes obtained by adding a His-tag to the sequence of a small subunit protein (S16, S10, S9, S8, or S6) on a plasmid which is over-expressed in E. coli.

Since all of these prior art methods employ plasmid based over-expression of a ribosomal component fused with the affinity tag, the success of these methods depends on the level of over-expression and also on the preferential integration of the over-expressed tagged component on the ribosome. Another unavoidable consequence is the contamination of the tagged component with the ribosome and therefore further purification steps are needed.

### SUMMARY OF THE INVENTION

The present invention solves the drawback with prior art methods by providing a general method for affinity tag based purification of any macromolecular complex present in the cell. In principle, any nucleotide sequence encoding an affinity-tag can be fused in frame with a gene at its chromosomal site. The gene should encode a regular component of the macromolecular complex which is present in multiple copies in the complex and preferably has well-exposed termini. As a result of this genetic engineering the macromolecular complex will carry the affinity tag on its surface, which can be employed for its purification.

Thus, in a first aspect the invention relates to a recombinant method to produce an affinity tagged macromolecular complex, comprising in-frame fusion of a nucleotide sequence specific for an affinity tag and a selection marker, wherein the fusion is at the chromosomal site of a gene encoding a multicopy protein, i.e. a protein present in the macromolecular complex in multiple copies. Thus, the macromolecular complex is expressed with multiple copies of said affinity tag. Preferably, the multicopy proteins are exposed at the surface of the macromolecular complex. This means that the affinity tag will be easily accessible for isolation/purifications purposes.

The macromolecular complex is preferably selected from replication complexes, transcription complexes, translation complexes, ribosomes, or any complex comprising multimeric functional molecules.

Preferably, the macromolecular complex is a ribosome and the gene is rplL comprising the nucleotide sequence disclosed in SEQ ID NO. 1 or any other sequence encoding rplL due to the degenerate nature of the genetic code.

This sequence encodes the prokaryotic multicopy protein L12 (also called L7/L12 in E. coli, L7 is the N-terminal acetylated form of the L12 protein). The present invention also relates to its homologues in bacteria, or its functional and compositional analogues (e.g. P1/P2 proteins) in eukaryotes. The detailed description of the prokaryotic L12 protein can be found on the Expasy website.

In a preferred embodiment the in-frame fusion is at the 3′-end of the gene's chromosomal site and is achieved by in-frame fusion of a linear sequence by recombination.

For selection purposes, preferably the linear sequence also comprises a marker gene. The marker gene may be, for example, a drug resistance gene; such as a kan- or amp- or tet- or cam- resistance cassette, or a lacZ, or other common markers appropriate for bacterial and/eukaryotic system.

The affinity tag is preferably inserted immediately before the stop codon in the gene for the ribosomal protein but may have other locations as well depending on the structure and location of the multicopy protein on the macromolecular complex.

Any affinity tag may be used according to the invention as long as it is small enough and will not interfere with the overall structure and function of the macromolecular complex. Examples of affinity tags are a His-tag, a FLAG-tag, Arg-tag, T7-tag, Strep-tag, S-tag, aptamer-tag, or any combination of these tags. Preferably, the affinity tag is a His₆-tag.

The affinity tag is used for affinity purification of the macromolecular complexes, such as ribosomes. Preferably, the macromolecular complexes are His-tagged ribosomes and the affinity purification method employs affinity chromatography. For His-tag complexes the affinity chromatography is preferably immobilized metal affinity chromatography (IMAC).

The method according to the invention enables purification of intact active 70S ribosomes but also of intact ribosomal subunits. The method may be used to purify ribosomes from wild-type as well as mutant strains.

In a preferred embodiment, the invention relates to a high-throughput single-step affinity-purification method of affinity tagged ribosomes, preferably tetra-(his)₆-tagged ribosomes from E. coli. The method of the invention is a quick and simple purification method resulting in a very high yield of the intact and active 70S ribosomes.

In a second aspect, the invention relates to affinity tagged ribosomes, comprising 4 copies of the L12 protein, or its homologue, which all are affinity tagged. Preferably, the tagged ribosomes are affinity tagged with at least two or more His-residues.

In a third aspect, the invention relates to a strain or cell line comprising the above described affinity tagged ribosomes. The strain or the cell line may of bacterial, yeast or plant origin.

In a fourth aspect, the invention relates to in vitro use of the above described affinity tagged ribosomes. A preferred use is for in vitro synthesis of proteins. Another use is for isolation/purification of translation complexes.

### BRIEF DESCRIPTION OF THE DRAWINGS

FIG. 1A is a strategy for designing the linear DNA cassette.

FIG. 1B is the insertion of the linear cassette at chromosomal site.

FIG. 1C is the verification of the linear cassette insertion by electrophoresis.

FIG. 2 is the comparison of growth rate between the strains MG1655 and JE28 in LB, 37° C.

FIG. 3 is the purification of the His₆-tagged ribosomes on His trap column as monitored by A₂₆₀.

FIG. 4A is the characterization of the His₆-tagged ribosomes by sucrose gradient analysis wherein the grey line represents the invention and black line represents prior art.

FIG. 4B is the characterization of the His₆-tagged ribosomes by 2D gel analysis; L7/L12 proteins are marked with white arrows. The reference protein L10 is marked by the black arrow to show the change in L12 position on the gel.

FIG. 4C is the characterization of the His₆-tagged ribosomes by ribosomal activity assay in dipeptide formation.

FIG. 5A is a subunit separation on His-trap column, imidazole elution profile.

FIG. 5B is a sucrose gradient analysis of the peaks obtained in FIG. 5A.

### DETAILED DESCRIPTION OF THE INVENTION

The present examples are provided for illustrative purposes only, and should not be construed as limiting the present invention as defined by the appended claims.

### Example 1

### Preparation of Linear DNA Cassette for λ Red Recombineering

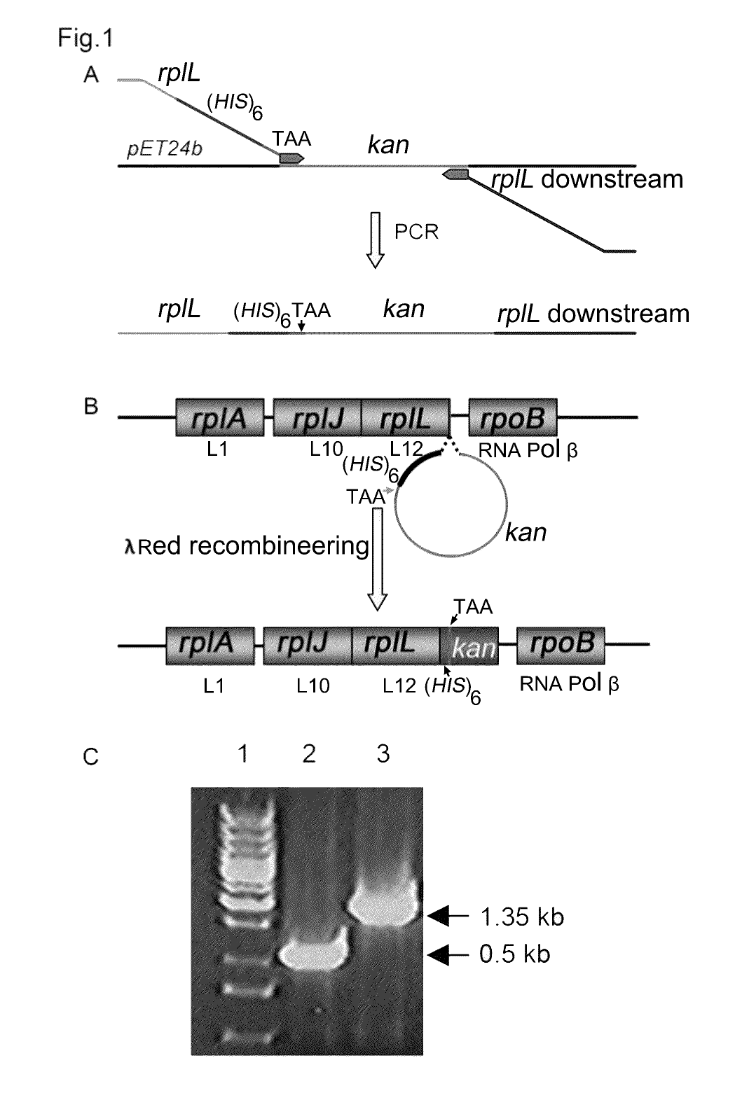

Standard PCR conditions were used to amplify the kanamycin-resistant cassette (kan) using pET24b plasmid (Novagen) as a template and two specially designed primers (FIG. 1A). The forward primer had the sequence (5′GAAAAAAGCTCTGGAAGAAGCTGGCGCTGAAGTTGAAGTTAAACACCACCAC CACCACCACTAAAAACAGTAATACAAGGGGTGTTATG-3′) (SEQ ID NO. 2) that contained 43 nucleotides homologous to the 3′-end of the E. coli rplL gene minus the stop codon, followed by six CAC repeats coding for six histidines, then stop codon TAA and 25 nucleotides homologous to the beginning of the kan cassette on the Novagen pET24 plasmid. The reverse primer (5′-ATCAGCCTGATTTCTCAGGCTGCAACCGGAAGGGTTGGCTTAGAAAAACTCA TCGAGCATCAAATGAAA-3′) (SEQ ID NO. 3) contained sequences, reverse complementary to 39 nucleotides located immediately after the rplL gene followed by the reverse complementary sequence to the last 30 nucleotides of the kan cassette of pET24b. It is note-worthy that in the primers the sequence homologous to 3′-end of the E. coli rplL gene and the sequence reverse complementary to downstream region of the rplL gene can vary in length between 30 and 55 with the optimal length around 40 nucleotides. These two sequences will constitute the DNA recombination (or more precisely the λ Red recombineering) site. Similarly, the length of the sequences used in the primers for annealing on the drug-cassette (kan-cassette) can be at least 10 and may vary in the higher side, depending on the total length of the primer. Both the primers were purchased from Invitrogen as custom synthesized and PAGE purified. The PCR product was purified from agarose gel using a commercial kit (Qiagen) and was used as a linear DNA cassette for λ Red recombineering.

### Example 2

### Construction of E. Coli Strains

Strain JE5 was constructed from E. coli HME6 strain (Costantino and Court, 2003; Ellis et al., 2001), where the stop codon of the rplL gene (coding ribosomal protein L12) was replaced by a linear PCR product encoding six histidines, a TAA stop codon followed by kanamycin-restistance cassette, using the λ Red recombineering system (Lee et al., 2001; Yu et al., 2000) (FIG. 1B). HME6 cells were made electroporation-competent and 1-2 μl of high quality PCR product (200-400 ng/μl) was added to 100 μl electro-competent HME6 cells and electroporated at 1.8 kV, 25 μF, and 200Ω. The electroporated cells were incubated overnight in 1 ml LB at 30° C. with aeration. Successful chromosomal recombinant colonies were selected on kanamycin plates and were confirmed by PCR with primers homologous to the flanking regions of the target site (FIG. 1C). Further, the C-terminus of rplL gene from some of the recombinant colonies was sequenced to confirm the correct insertion of his₆ tag at the C-terminus of L12. The ones with the desired insertion were named JE5. Further the his-tagged rplL gene was transferred from JE5 to the wild type lab strain MG1655 using standard protocols by generalized transduction with bacteriophage P1 yeilding a new stable E. coli strain JE28. JE28 strain bears kanamycin resistance and the sequencing of C-terminus of rplL gene from it confirmed the endogenous insertion of the his-tag at the C-terminal of L12. The genotypes of the strains used in the invention are listed in Table 1.

TABLE 1
Genotype of E. coli strains

| Strains | Genotype |
|---|---|
| HME6 | W3110, Δ(argF-lac)U169 |
| | gal⁺ {λcI857 Δcro-bioA} galK_{TYR145UAG} |
| JE5 | HME₆, rplL-his₆::Kan^{R} |
| MG1655 | pyrE⁺ |
| JE28 | MG1655, rplL-his₆::Kan^{R} |

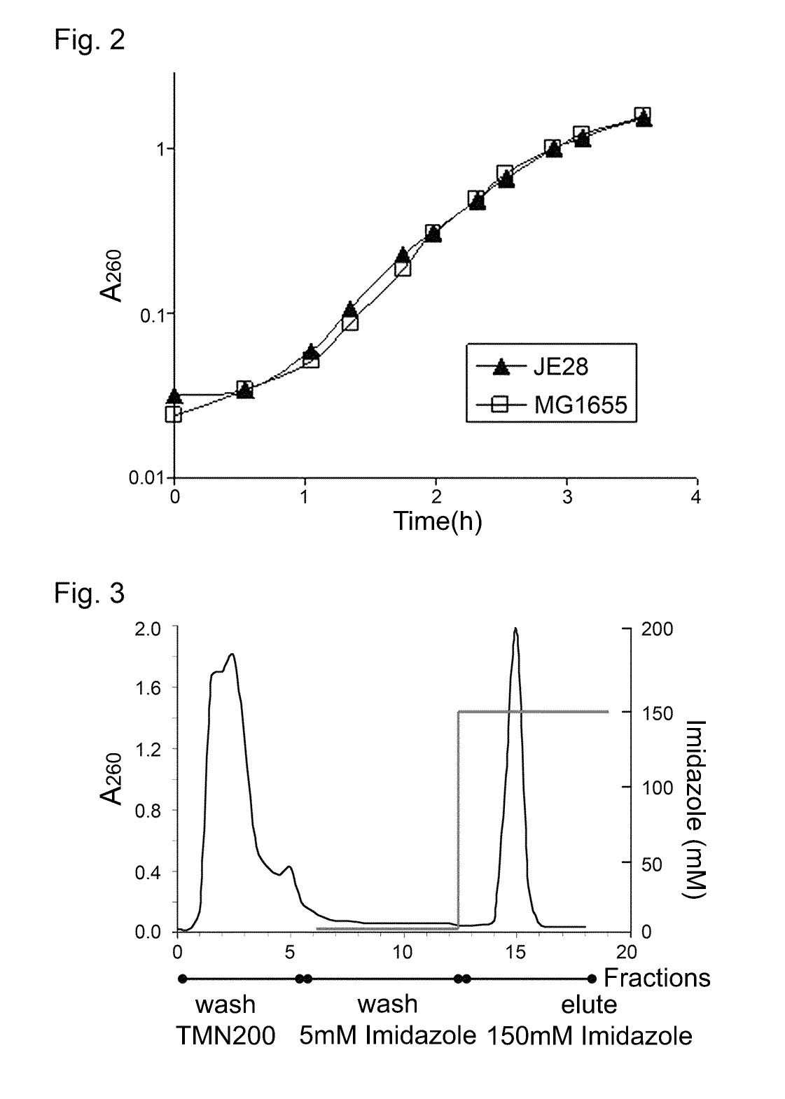

To compare the growth rate of JE28 with the parental strain MG1655, both the strains were grown in LB at 37° C., and the absorbance at 600 nm was monitored (FIG. 2). For JE28, the assay was repeated in the presence of kanamycin (50 μg/ml), which had essentially no effect on its growth rate.

### Example 3

### Purification of his-Tagged Ribosomes

To purify the tetra-His₆-tagged ribosomes, JE28 was grown in LB at 37° C. to A₆₀₀ ˜1.0, slowly cooled to 4° C. to produce run off ribosomes and pelleted. The cells were resuspended in lysis buffer (20 mM Tris-HCl pH 7.6, 10 mM MgCl₂, 150 mM KCl, 30 mM NH₄Cl, and PMSF protease inhibitor 200 μl/l) with lysozyme (0.5 mg/ml) and DNAse I (10 μg/ml) and lysed using a French Press or sonicator (for smaller cell pellets <2-3 g). The lysate was clarified twice by centrifugation for 20 min at 18,000 rpm at 4° C. The lysate was divided into two equal halves and 70S ribosomes were purified with the conventional method (A, below) from one half whereas the affinity-purification method (B, below) was used on the other half. In parallel, ribosome from the parent strain MG1655 was also purified in the conventional way for comparison.

### A: Conventional Method

For purifying JE28 ribosomes in a conventional method the cleared lysate was layered on top of equal volume of 30% w/v sucrose cushion made in the buffer (20 mM Tris-HCl pH 7.6, 500 mM NH₄Cl, 10.5 mM Mg Acetate, 0.5 mM EDTA, and 7 mM 2-mercaptoethanol) and centrifuged at 100,000 g for 16 hours at 4° C. This step was repeated twice and in between the pellet was gently rinsed with the same buffer. The final ribosome pellet was treated in the same way as the affinity purified ribosomes for storage or sucrose gradient analysis. In parallel, MG1655 70S ribosomes are also prepared in the conventional way.

### B: Affinity Purification According to the Invention

For affinity purification a HISTRAP™ HP column (Ni²⁺SEPHAROSE™ pre-packed, 5 ml, GE Healthcare Bio-Sciences AB) was connected to an ÄKTA™ prime chromatography system (GE Healthcare Bio-Sciences AB) and equilibrated with the lysis buffer. After loading the lysate (2 ml/min), the column was washed with 5 mM imidazole in the lysis buffer for several column volumes until A₂₆₀ reached the baseline. His-tagged ribosomes were then eluted with 150 mM imidazole containing lysis buffer, pooled immediately and dialyzed 4×10 minutes in 250 ml lysis buffer. After dialysis the ribosomes were concentrated by centrifugation at 150,000 g for two hours at 4° C. and resuspended in 1×polymix buffer containing 5 mM ammonium chloride, 95 mM potassium chloride, 0.5 mM calcium chloride, 8 mM putrescine, 1 mM spermidine, 5 mM potassium phosphate and 1 mM dithioerythritol and shock-froze in liquid nitrogen for storage or dissolved in the overlay buffer (20 mM Tris-HCl pH 7.6, 60 mM NH₄Cl, 5.25 mM Mg Acetate, 0.25 mM EDTA, and 3 mM 2-mercaptoethanol) for sucrose gradient analysis. As a control system, lysate from wild type E. coli strain MG1655 was applied in the same column and was treated accordingly, but no ribosome was found in the elute.

### Example 4

### Sucrose Gradient Analysis of Ribosomes

The his-tagged ribosomes from JE28 purified by the affinity method were assessed for the subunit composition by sucrose gradient analysis. 3000 pmol of ribosomes were loaded on a 20-50% sucrose density gradient (18 ml) prepared in a buffer containing 20 mM Tris-HCl pH 7.6, 300 mM NH₄Cl, 5 mM Mg Acetate, 0.5 mM EDTA, and 7 mM 2-mercaptoethanol and centrifuged at 100,000 g for 16 hours at 4° C. For comparison, JE28 ribosomes prepared in the conventional way were also analyzed in parallel. E. coli MG1655 ribosomes and subunits prepared in the conventional way were used as standards.

Two dimensional gel analysis of the purified ribosomes was performed for the ribosomes produced according to the invention and for the conventionally produced ribosomes.

### Example 5

### Activity of the Purified Ribosomes in Dipeptide Formation Assay

The dipeptide assay was designed following the protocol described by Antoun et al. for dipeptide fMet-Phe (Antoun et al., 2006), with modifications necessary for the formation of dipeptide fMet-Leu. The components which were specially needed for modification of this assay included an mRNA coding for fMet-Leu-Stop, tRNA aminoacyl synthetase LeuRS, tRNA^{Leu} and the amino acid Leu, instead of fMet-Phe-Thr-Ile-stop mRNA, PheRS, tRNA^{Phe} and the amino acid Phe used by Antoun et al. respectively. Instead of using the LS-buffer used by Antoun et al., the assay was performed in 1×polymix buffer described above.

### Example 6

### Purification of Ribosomal Subunits from JE28 Ribosomes

For purification of the ribosomal subunits employing the affinity method, the his₆-tagged ribosome was dialysed or diluted in low-Mg buffer containing 20 mM Tris-HCl pH 7.6, 1 mM MgCl₂, 150 mM KCl and 30 mM NH₄Cl and was loaded on a HISTRAP™ HP column equilibrated with the same buffer. Since the his₆-tag was on the 50S subunit, the 30S subunits were not retained on the column and were collected in the flow-through. The his₆-tagged 50S subunits were eluted from the column and the subunits were concentrated following the same procedure as described above for the his₆-tagged 70S ribosomes.

For separation of ribosomal subunits in the conventional way, 70S ribosomes were dialyzed in low-Mg buffer containing 20 mM Tris-HCl pH 7.6, 300 mM NH₄Cl, 3 mM Mg Acetate, 0.5 mM EDTA, and 7 mM 2-mercaptoethanol and separated by ultra-centrifugation (85,000 g at 4° C. for 16 h) on 20-50% sucrose density gradients (18 ml) prepared in the same buffer. The gradients were fractionated monitoring the absorbance at 260 nm. Respective peak fractions for 50S and 30S were pooled, concentrated by centrifugation at 150,000 g for two hours at 4° C., resuspended in 1×polymix buffer and stored in the same as described above for 70S ribosomes.

### Results

The E. coli strain JE28 has an in-frame fusion of a nucleotide sequence encoding a hexa-histidine affinity tag at the 3′-end of the single copy rplL gene (coding ribosomal protein L12) at its chromosomal site followed by the insertion of a kan cassette (−800 nucleotides) as a marker gene. The total length of the inserted sequence was about 850 nucleotides. JE28 was successfully grown on kan-LB plates for several generations when the stability of the inserted sequence was verified by its kanamycin resistance as well as checked by PCR using primers flanking the rplL gene (FIG. 1C). When compared with MG1655, it showed essentially the same growth rate in liquid culture (LB) (FIG. 2) irrespective of the presence of kanamycin (data not shown). This result confirms the following. First, the targeted insertion at the chromosomal site was stable and did not affect the expression of the genes located further downstream on the same operon (e.g. rpoB coding for the beta-subunit of RNA polymerase) (FIG. 1B). Second, the his₆-tags inserted on the C-termini of L12 proteins on the 50S subunit of the ribosome did not interfere with the ribosome function in vivo and more specifically with the function of the ribosomal ‘stalk’ protein L12. This has been tested further in vitro.

A novel affinity-tag based method for the purification of E. coli ribosomes was developed making use of a his₆-tag inserted stably on the C-termini of the L12 proteins on the large subunit of the ribosome in E. coli JE28. FIG. 3 describes the elution profile from the HISTRAP™ HP column (GE Healthcare Bio-Sciences AB) monitored as a function of absorbance at 260 nm. The peak fractions eluted with 150 mM imidazole showed a A₂₆₀/A₂₈₀ ratio of 1.9, a value typical for ribosome. These fractions were pooled, concentrated and were subjected to further analysis by sucrose density gradient centrifugation, 2D gel and activity assay in dipeptide bond formation. In a control experiment with the lysate from MG1655, no significant peak was eluted from the HISTRAP™ HP column and the pooled peak fractions did not show any nucleic acid specific absorbance at 260 nm (data not shown).

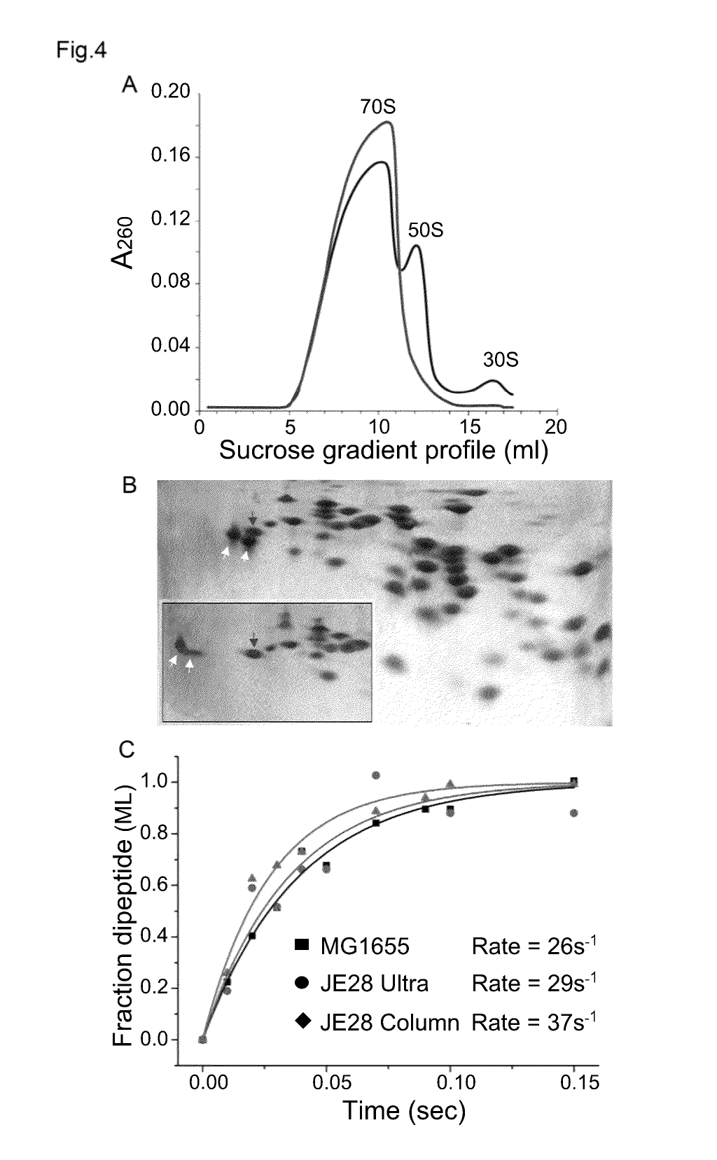

The JE28 ribosomes purified in the affinity method as well as in the conventional method were subjected to sucrose density gradient centrifugation analysis. Under the above described buffer conditions, the affinity purified ribosomes contained only 70S ribosomes whereas the ribosomes purified in the conventional way contained 70S as well as 50S and 30S subunits (FIG. 4A).

The yield of pure 70S ribosomes in the affinity purification method was much higher compared to the conventional purification method. This is due to the fact that pure 70S ribosomes could be obtained directly from one-step HISTRAP™ HP column elution in the affinity purification method, whereas in the conventional method purification of 70S ribosomes needed additional sucrose density gradient ultracentrifugation.

The his₆-tagged JE28 70S ribosomes purified on a HISTRAP™ HP column was characterized in 2D-gel (FIG. 4B). In parallel, 70S ribosomes from MG1655 were also subjected to 2D-gel analysis for comparison (FIG. 4B, inset). All the 52 ribosomal proteins were identified in identical positions on the gel in both the samples with the exception of L12 (L7/L12) proteins (indicated by white arrows in FIG. 4B), which were moved from their original position due to the insertion of the his₆-tags. This is seen clearly when their position was compared with another ribosomal protein L10 (indicated by the black arrow in FIG. 4B). L12 is a highly acidic protein (pI 4.6) and L7 is the N-terminal acetylated form of L12. The addition of six basic Histidine residues to L12 resulted in a changed pI (5.2) of the protein and caused the change of the position on the 2D gel.

Peptide bond formation is central to ribosome functions. In a cell-free translation system composed of purified components from E. coli, tetra-(his)₆-tagged JE28 ribosomes purified in the affinity method (JE28 Column in FIG. 4C) showed faster rate of dipeptide (fMet-Leu) formation when compared to the JE28 as well as MG1655 ribosomes purified in the conventional way (referred as JE28Ultra and MG1655 respectively in FIG. 4C). This result confirmed that the chromosomal insertion of the tetra-(his)₆-tag on the C-termini of L12 proteins did not affect negatively the ribosomal function in translation factor associated peptide bond formation. The higher activity in dipeptide formation could be due to the higher homogeneity of the 70S ribosomes purified in the affinity method. The ribosomes purified in the conventional way by ultracentrifugation contained some free 50S and 30S subunits together with 70S as evidenced in sucrose gradient analysis (FIG. 4A).

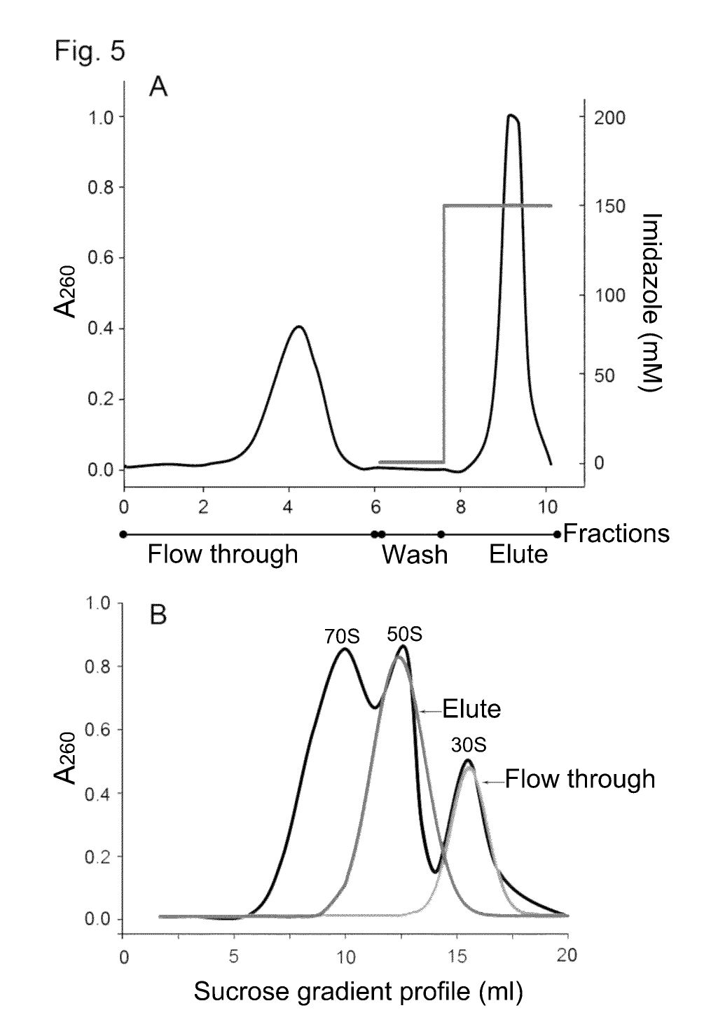

The presence of the tetra-(his)₆-tag only on the 50S subunit, but not on the 30S subunit enabled us to develop a method for purification of ribosomal subunits using the HISTRAP™ HP column in low-Mg⁺² buffer. FIG. 5A represents the elution profile of the column with two distinct peaks. The first peak (flow-through) when pooled and analyzed in sucrose gradient analysis showed only 30S subunits and the second peak eluted with 150 mM imidazole was identified as 50S subunits (FIG. 5B).

### Applications of the Tagged Ribosomes According to the Invention

The tetra-his₆-tagged ribosomes can be used to isolate functional translation complexes bound with mRNA, tRNA, translation factors, nascent protein chain and/or other ribosome associated proteins such as chaperones. Now that the structure and function of the bacterial ribosome are known in molecular details there is a growing demand for the structural studies of ribosomal complexes trapped in different functional steps by cryo-EM or X-ray crystallography. Using the affinity tag on the ribosome and appropriate physiological buffer conditions functional complexes can be directly isolated with the factors adhered on the ribosome.

The E. coli strains which carry mutations in the ribosomal RNA or protein genes often contain small amount of ribosomes in the cell and is therefore difficult to purify with good yield by conventional method. For purification of ribosomes from these mutant strains, the affinity tag with the drug marker can be moved from JE28 to the respective mutant strains by generalized transduction with bacteriophage P1 and then the affinity method of purification of JE28 ribosomes can be followed.

The affinity purified ribosomes can be added to the ‘cell-lysate’ based in vitro protein synthesis systems to increase the efficiency of protein production from these systems.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A method to produce an affinity tagged macromolecular complex, comprising in-frame fusion of a nucleotide sequence comprising an affinity tag and a selection marker, wherein the fusion is at the chromosomal site of a gene encoding a multicopy protein.

2. The method of claim 1, wherein the multicopy protein is exposed at the surface of the macromolecular complex.

3. The method of claim 1, wherein the macromolecular complex is selected from replication complexes, transcription complexes, translation complexes, ribosomes, or any complex comprising multimeric functional molecules.

4. The method of claim 1, wherein the macromolecular complex is a ribosome and the multicopy protein is prokaryotic L12 or its homologues.

5. The method of claim 4, wherein the in-frame fusion is at the 3′-end of the gene's chromosomal site and is achieved by in-frame fusion of a linear sequence by recombination.

6. The method of claim 1, wherein the selection marker gene is a drug-resistance gene.

7. The method of claim 1, wherein the affinity tag is inserted immediately before or close to the stop codon in the gene encoding the multicopy protein, which retains its function.

8. The method of claim 1, wherein the affinity tag is selected from a His-tag, a Flag tag, Arg-tag, T7-tag, Strep-tag, S-tag, aptamer tag, or any combination of these tags thereof.

9. The method of claim 8, wherein the affinity tag is a His₆-tag.

10. The method of claim 1, comprising affinity purification of the macromolecular complexes, such as ribosomes, using said affinity tag.

11. The method of claim 10, wherein the ribosomes are His-tagged and the affinity purification method is affinity chromatography.

12. The method of claim 11, wherein the affinity chromatography is immobilized metal affinity chromatography (IMAC).

13. The method of claim 10, wherein the intact active 70S ribosomes are purified.

14. The method of claim 10, wherein intact ribosomal subunits are purified.

15. Affinity tagged ribosomes, comprising multiple copies of the prokaryotic L12 protein, or its homologue, which all are affinity tagged.

16. The affinity tagged ribosomes of claim 15, which are affinity tagged with two or more His-residues.

17. A cell comprising the affinity tagged ribosomes of claim 15.

18. The cell of claim 17, wherein the cell has one or more mutations in its ribosomal genes.

19-21. (canceled)


SEQUENCE LISTING (3 sequences)

SEQ ID NO: 1 - DNA, 366 bp, Escherichia coli
atgtctatcactaaagatcaaatcattgaagcagttgcagctatgtctgtaatggacgttgtagaactgatctctgcaatggaagaaaaattcggtgtttccgctgctgctgctgtagctgtagctgctggcccggttgaagctgctgaagaaaaaactgaattcgacgtaattctgaaagctgctggcgctaacaaagttgctgttatcaaagcagtacgtggcgcaactggcctgggtctgaaagaagctaaagacctggtagaatctgcaccggctgctctgaaagaaggcgtgagcaaagacgacgcagaagcactgaaaaaagctctggaagaagctggcgctgaagttgaagttaaataa

SEQ ID NO: 2 - DNA, 89 bp, Artificial Sequence
  Forward primer sequence
gaaaaaagctctggaagaagctggcgctgaagttgaagttaaacaccaccaccaccaccactaaaaacagtaatacaaggggtgttatg

SEQ ID NO: 3 - DNA, 69 bp, Artificial Sequence
  Reverse primer sequence
atcagcctgatttctcaggctgcaaccggaagggttggcttagaaaaactcatcgagcatcaaatgaaa